(19) United States
(12) Patent Application Publication
(10) Pub. No.: US 2010/0042054 A1
(43) Pub. Date: Feb. 18, 2010

(54) Medical Delivery System Comprising a Coding Mechanism Between Dosing Assembly and Medicament Container

(75) Inventors: Ramin Nateghi Elahi, Gorlose (DK); Jonas Torry-Smith, Copenhagen (DK); Michael Ejstrup Hansen, Morud (DK); Nikolaj Eusebius Jakobsen, Valby (DK); Thomas Pedersen, Helsingoer (DK); Thomas Geertsen, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/513,041

(22) PCT Filed: Nov. 17, 2007
(86) PCT No.: PCT/EP2007/062478

§ 371 Date: Sep. 3, 2009

(30) Foreign Application Priority Data
Nov. 17, 2006 (EP) 06023880.5

(51) Int. Cl. A61M 5/315

(52) U.S. Cl. 604/211; 604/208

(57) ABSTRACT

A medical delivery system comprising a container for accommodation of a medicament and a dosing assembly adapted to be fastened to the container. The medical delivery system comprises a coding mechanism (150, 152) for ensuring that only a predetermined container can be fastened to a predetermined dosing assembly. The coding mechanism comprises axially extending protrusions (154, 156, 158) arranged at the interface between container and dosing assembly and arranged in a ring configuration concentric with a proximal rim portion of the container. A container for use in the medical delivery system. A dosing assembly for use in the medical delivery system.

### FIELD OF THE INVENTION

The present invention relates to a medical delivery system wherein a container may be fastened to a dosing assembly by engagement between a first and a second fastening means. Moreover, the present invention relates to a medical delivery system comprising a first and second container and a first and second dosing assembly. In particular the present invention relates to a medical delivery system comprising a container and dosing assembly with a coding and coupling system. Moreover, the present invention relates to a container and a dosing assembly for use in the medical delivery system.

### BACKGROUND OF THE INVENTION

Generally, in order to provide superior medication delivery devices which are likely to be well received by particular groups of patients, a greater diversity in drug delivery systems have been launched to the benefit of patients. As the number of commercially available delivery systems increase, numerous different types of medication holding cartridges or containers are distributed. Most of these types of containers differ in various aspects.

Each medicament container may be filled with a particular type of medicament selected from a large variety of different medicaments, but also different kinds of the same class of medicament (e.g. rapid or long acting insulin) and different concentrations of each particular medicament may be accommodated in the containers.

Moreover, different container volumes may be introduced in order to customize each container, and, thus, the delivery system to the needs of particular users. Variation of container volume may be provided by changing the length or diameter of the container. These modifications usually imply corresponding modifications of the dosing assembly of a medication delivery system, so as to provide a particular stroke of a driving element for expelling the medicament from the container or to provide optimal dosing precision. Further discrimination between different medicament containers may be occasioned by the design requirements for each particular delivery system, such as required sliding friction of the piston accommodated in the container.

In order to discriminate between a larger variety of available containers, numerous container coding and coupling systems have been developed. The following mechanical coding and coupling systems are known in the art:

U.S. Pat. No. 5,611,783 relates to a pen shaped syringe comprising a distal part which may comprise an ampoule and a proximal part containing a dose setting and drive mechanism. The proximal and distal parts have interlocking bayonet coupling means. Protrusions may be provided to form a pattern ensuring that a certain distal part may only be used in connection with a certain proximal part.

WO 03/017915 A1 discloses a cartridge having a distal end provided with a mechanical coding. The mechanical coding has the form of a circular protrusion where the circular outer diameter is dedicated a specific concentration of insulin contained in the cartridge.

U.S. Pat. No. 5,693,027 discloses a plastic top for adapting a standard cartridge to a chosen syringe. The plastic top may be provided with means for keyed engagement with corresponding means in a syringe to keep it unrotatable when mounted with a cartridge in the syringe. In some types of syringes such keyed engagement between cartridge and syringe is further used to ensure that only a certain type of cartridge is used.

U.S. Pat. No. 6,648,859 B2 discloses a drug cartridge assembly for use with a reusable pen body assembly of a medication delivery pen. In order to eliminate cross-use the pen body assembly and the drug cartridge are keyed i.e. they may be threadedly engaged by corresponding threads and grooves, bayonet threads, and grooves, snap fits or a pair of lugs that mate in reverse Luer-Lock manner. The mating members are selected so as to prevent cross-use with other assemblies, e.g., the pitch of the threads may be angled so as to mate only with one another and not with other assemblies.

Yet other prior art systems are described in DE 201 10 690, WO 03/011372 and WO 03/011373.

To the extent that the above described references relate to the elimination of cross-use between sets of distinct containers and their respective compatible dosing assemblies, the referenced art remains somewhat flawed having regard to the risk of mechanical failure. In particular, this is the case where the coupling movement required for coupling and locking a container to a related dosing assembly occurs by a movement including a relative rotational movement.

It is an object of a preferred embodiment of the present invention to provide an alternative to the known systems. Furthermore, it is an object of a preferred embodiment of the present invention to provide a medication delivery system with a large number of possible coding geometries while yet, compared to prior art systems, provides a robust coding geometry.

In addition, it is an object of a preferred embodiment of the present invention to provide a coding system where rotational alignment between containers and compatible dosing assemblies are not required when coupling the container to the dosing assembly.

Furthermore, it is an object of a preferred embodiment of the present invention to provide a coding system wherein the user experiences substantially the same operational fastening/coupling/locking movement when the container and dosing assembly of a predetermined medical delivery system are coupled/uncoupled (locked/unlocked) to each other regardless of the specific choice among sets of compatible container/dosing assemblies.

### SUMMERY OF THE INVENTION

In a FIRST aspect the present invention relates to a medical delivery system comprising:
a container adapted to contain a medicament in a chamber defined by the container and a slidably arranged piston which is axially moveable in a distal direction towards an outlet so as to reduce the volume of the chamber and expel the medicament through the outlet, the container comprising a housing section having a proximal cylindrical rim section which defines a proximal internal opening;
a piston rod adapted to extend through said proximal internal opening of the container and move the piston in the distal direction;
a dosing assembly adapted to be fastened to the container so as to bring the dosing assembly into operational contact with the container, the dosing assembly comprising a driver which when actuated is adapted to move the piston rod in the distal direction when the dosing assembly is in said operational contact with the container;
wherein the dosing assembly defines first fastening means adapted to engage second fastening means of the container so as to lock the container in said operational contact with the dosing assembly;
wherein the proximal end part of the container defines at least one coding surface dedicated the medicament to be contained in the container, the coding surface comprising one or more proximally extending protrusion(s), the or each proximally extending protrusion(s) being arranged in a first ring configuration concentric with said rim portion;
wherein the dosing assembly defines a coding surface dedicated the medicament to be contained in the container and comprising one or more distally extending protrusion(s), the or each distally extending protrusion(s) being arranged in a second ring configuration; and

wherein the first and the second ring configurations are adapted to mesh and axially overlap when the container is locked in operational contact with the dosing assembly.

In the context of the present invention the term “medical delivery system” shall be understood as any system capable of administering a medicament-containing flowable drug. Examples of medical delivery systems are infusion pump applications, dosers, pen-shaped dosers, motor-dosers, and automated syringes such as the AutoPen(™).

The invention is applicable to all kinds of medicament delivery devices capable of delivering a medicament to a user from a container which is adapted to be coupled to a dosing assembly of the delivery device. The delivery device may include any delivery device for transcutaneous, subcutaneous, intravenous, intra muscular or pulmonary administration of a drug.

As used herein, the term “medicament” is meant to encompass any medicament-containing flowable drug capable of being passed through a delivery means such as a hollow needle in a controlled manner, such as a liquid, solution, gel or fine suspension. Representative medicaments includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The chamber of the container may be defined by one or more sidewalls of the container and the slidably arranged piston. In most embodiments at least a part of the container is ring-shaped and defines a cylindrical cavity in which the piston is received. The distal end of the container may comprise a seal for penetration by a cannula so as to allow a medicament contained in the chamber to be expelled through the cannula. The distal end of the container may be adapted to be attached to a holder holding a cannula. As an example the distal end of the container may comprise a thread adapted to cooperate with a corresponding thread of the holder so as to allow the holder to be screwed onto the container.

The outlet of the container may be adapted to cooperate with or be defined by a cannula or a needle or a needle hub or an infusion set, or any other fluid communicating conduit adapted to provide fluid access to a medicament accommodated in the container.

The piston rod may comprise an element which is more rigid than the piston and is adapted to abut at least a part of and preferably most of the proximal facing surface of the piston whereby a force applied by the piston rod to the rigid element is applied to a larger area of the proximal surface of the piston than if the piston rod had engaged the piston directly. This is advantageous when the piston is made of a resilient material.

In the context of the present invention the terms “depression”, “groove” and “projection” are only used in connection with radially extending members/elements/means, and “indentation” and “protrusion” are only used in connection with axially extending members/elements/means. However, “depression”, “groove” and “indentation” shall be seen as synonyms and “protrusion” and “projection” shall be seen as synonyms.

The invention according to the first aspect improves user safety as only predetermined containers may be attached to predetermined dosing assemblies. Thus, the dosing assembly may be designated to be used with a predetermined kind and/or concentration of a medicament and non-compatible containers accommodating other concentrations or types of medicaments cannot be attached to the dosing assembly. As the coding surfaces of the container and the dosing assembly are provided as ring configurations which are arranged concentrically, a robust coding system is provided, which thus increases safety.

The medical delivery system may further comprise:
a first container according to the first aspect of the invention, the container being adapted to be fastened to a first dosing assembly according to the first aspect of the invention,
a second container according to the first aspect of the invention, the container being adapted to be fastened to a second dosing assembly according to the first aspect of the invention,

wherein the first and/or the second coding mechanism of at least two of the first container, the second container, the first dosing assembly and the second dosing assembly, is/are adapted to prevent the first dosing assembly and second container from being fastened to each other, and to prevent the second dosing assembly and the first container from being fastened to each other.

The first container may be coupled to the first dosing assembly through a predetermined movement which is substantially identical to the predetermined movement required for coupling the second container to the second dosing assembly.

The system may be so adapted that relative rotation between a container and its compatible dosing assembly is allowed during the entire procedure for coupling the container to the dosing assembly, i.e. also while the container axially approaches its position where the container is locked in operational contact with the dosing assembly.

The container and the dosing assembly may be adapted to be mutually locked by a movement comprising a relative rotational movement, e.g. by a threaded connection or by a bayonet lock type connection.

Alternatively, the container and the dosing assembly is adapted to be mutually locked through a translational and non-rotational movement. Such locking procedure may be arranged where relative rotational movements may be performed during axial coupling and locking, but where the relative rotational movement is not a prerequisite.

When an extreme proximal portion of an external wall section of the container housing defines a rim part encircling a proximal opening, the axially extending protrusions may be totally or partly accommodated in the opening. In such case, the rim portion of the container protects the coding surface from mechanical impact. Especially in case of fine-detailed coding structures, this is advantageous. In other embodiments, the axially extending protrusions may extend beyond the opening in the proximal direction.

The container may be formed as a unitary housing which holds the piston and the medicament in fluid contact with the wall sections of the housing. Such container may be manufactured as a molded article having the axial extending protrusions formed integrally by molding. Alternatively, the container functions as a cartridge holder, where a medicament glass cartridge is partly of fully accommodated in the cartridge holder. The cartridge may be removably attached to the cartridge holder, or alternatively, permanently attached to or in the cartridge holder. The axially protrusions of the container may be formed unitarily with the cartridge holder or alternatively attached to the cartridge holder as a separate element. As such, the axially extending protrusions are formed by an element which is separate from the glass cartridge which allows for the fine-detailed structure of the coding surface.

In a SECOND aspect the present invention relates to a medical delivery system comprising:
a container adapted to contain a medicament in a chamber defined by the container and a slidably arranged piston which is moveable in a distal direction towards an outlet so as to reduce the volume of the chamber and expel the medicament through the outlet;
a dosing assembly adapted to be fastened to the container so as to bring the dosing assembly into operational contact with the container, the dosing assembly comprising a driver adapted to move the piston in the distal direction when the dosing assembly is in said operational contact with the container;
wherein one of the dosing assembly and the container defines first fastening means adapted to engage second fastening means of the other one of container and the dosing assembly so as to lock the container in said operational contact with the dosing assembly; and
wherein the second fastening means defines a radially extending groove and the first fastening means comprises a flexible locking member which may be changed between:
   a locking shape wherein at least a part of the locking member engages at least a part of the radially extending groove whereby the container is locked for translational movement relative to the dosing assembly, and
   a non-locking shape wherein the locking member does not engage the radially extending groove whereby the container is not locked for translational movement relative to the dosing assembly, the non-locking shape being different from the locking shape.

The radially extending groove may be defined on the outer surface of the container and may extend around the entire circumference of the container so as to define a circumferentially extending groove. Alternatively, the radially extending groove may be split into groove parts spaced along the outer circumference of the container. In one embodiment the locking member is adapted to engage a part of the groove during locking/fastening of the container to the dosing assembly.

In one embodiment the container comprises the second fastening means and the dosing assembly comprises the first locking means. In an alternative embodiment, the dosing assembly comprises the second fastening means and the container comprises the first fastening means.

In one embodiment the locking member is adapted to change shape from the locking shape to the non-locking shape, by application of finger pressure to predetermined areas of the locking member such as to two buttons provided on opposite sides of the locking member, and in the reverse direction when the finger pressure is removed.

In one embodiment the locking member comprises a resilient material biasing the locking member towards the locking position. The resilient material may be any material suitable to be reshaped by application of finger pressure, such as rubber, soft plastics, spring steel. In one embodiment the locking member comprises at least two buttons which when the locking member is assembled in the dosing assembly are accessible from an outer surface of the dosing assembly. Application of finger pressure to the buttons (such that the two buttons are moved towards each other) causes the locking member to change from the locking to the non-locking shape whereby a user may remove the container from the dosing assembly. Moreover, at least a part of the locking member may be substantially elliptical when defining the locking shape and substantially cylindrical when defining the non-locking shape.

Furthermore, one of first and second fastening means may define one or more radially extending projection(s) which during fastening is adapted to be received in one or more corresponding radially extending depressions of the other one of the first and second fastening means. Said projections/depressions may define coding surfaces as described in relation to the invention according to the third aspect.

In a further embodiment one of the first and second fastening means comprises a plurality of radially and axially extending projections such as two, three, four, five, six, seven, eight, nine or ten and the other one of the first and second fastening means comprises a corresponding number of depressions or grooves. The coding surfaces of the radially extending projections may be defined by the radial and/or axial and/or circumferential extent of each of the projections and the relative circumferential position of the projections on an inner or outer surface of the first or second fastening means.

The radially extending projections may be defined in a symmetrical or asymmetrical pattern in a plane transverse to the longitudinal axis of the device.

In the context of the present invention the term “asymmetrical pattern” shall be understood such that when the radially extending projections define an asymmetrical pattern, there exist only one relative rotational position between two elements e.g. the first and second fastening means, wherein the radially extending projections may be received in the corresponding radially extending depressions.

In a THIRD aspect the present invention relates to a medical delivery system comprising:
a container adapted to contain a medicament in a chamber defined by the container and a slidably arranged piston which is moveable in a distal direction towards an outlet so as to reduce the volume of the chamber and expel the medicament through the outlet;
a piston rod adapted to move the piston in the distal direction;
a dosing assembly adapted to be fastened to the container so as to bring the dosing assembly into operational contact with the container, the dosing assembly comprising a driver adapted to move the piston rod in the distal direction when the dosing assembly is in said operational contact with the container;
wherein the dosing assembly defines a first fastening means adapted to engage a second fastening means of the container so as to lock the container in said operational contact with the dosing assembly, the second fastening means being defined on a proximal end part of the container; and
wherein the proximal end part defines at least one coding surface adapted to cooperate with a corresponding coding surface of the dosing assembly so as to prevent the dosing assembly and the container from being locked in the operational contact unless each of the coding surfaces are selected from a predetermined group of coding surfaces.

The piston rod may be rotationally fixed relative to the proximal end part while being allowed to move axially relative to said proximal end part. Furthermore, the container and/or the proximal end part may comprise the piston rod. In one embodiment the piston rod is detachably attached to the proximal end part and/or the container. In another embodiment the piston rod is retained relative to the proximal end part and/or the container, such that the piston rod cannot be removed from the container/proximal end part. As an example the proximal end part and/or the container may limit translational movement of the piston rod relative to the proximal end part and/or the container, such that the piston rod only can be moved between a first and a second position relative to the proximal end part. The piston rod may be slidingly received in the proximal end part and/or the container, such that the piston rod may slide relative to the proximal end part and/or the container between two positions.

In one embodiment, a sidewall of one of the proximal end part and the dosing assembly defines at least one projection extending in a radial direction, each of the at least one projection being adapted to engage a corresponding radially extending depression or groove defined in a sidewall of the other one of the proximal end part and the dosing assembly, so as to lock the proximal part and the dosing assembly for relative rotational movement when the container is in operational contact with the dosing assembly. Moreover, the at least one radially extending projection and/or corresponding depression may define at least one of the coding surfaces.

In one embodiment the container comprises a plurality of radially and axially extending projections such as two, three, four, five, six, seven, eight, nine or ten and the dosing assembly comprises a corresponding number of depressions or grooves. The coding surfaces of the radially extending projections may be defined by the radial and/or axial and/or circumferential extent of each of the projections and the relative circumferential position of the projections on an outer surface of the container or an inner surface of the dosing assembly. Accordingly, two radially extending projections may have different radial extent and/or axial extent or circumferential extent. Moreover, the radial or axial extent of each projection may vary e.g. such that the radial extent of a projection decreases in a proximal direction of the device.

The radially extending projections may be defined in a symmetrical or asymmetrical pattern in a plane transverse to the longitudinal axis of the device.

It will be appreciated that in some embodiments of the medical delivery system, one dosing assembly may be adapted to be fastened to a plurality of predetermined containers. As an example the dosing assembly may comprise eight radially extending projections positioned in eight predetermined positions, and thus any container having projections in one or more of said eight predetermined positions and not in any other positions, will be adapted to be connected to the dosing assembly. Thus, a container having five radially extending projections in five of the eight positions, may be coupled to the dosing assembly provided that the circumferential/radial/axial extent/position of the eight depressions/grooves of the dosing assembly allow the said five projections to be received in the depressions/grooves.

The coding surfaces may be defined on one of the first and/or second fastening means, such as on a radially and/or an axially and/or a circumferentially extending surface of the first/second fastening means. In one embodiment, the first fastening means of the dosing assembly is adapted to be moved radially into engagement with the second fastening means (e.g. a groove) of the container so as to lock the container to the dosing assembly. In the latter embodiment, a projection may be defined in the groove, for preventing the first fastening means from engaging the groove unless the first fastening means defines a corresponding depression for receiving the projection of the groove of the container.

Additionally or as a supplement, the proximal part of the container may define at least one proximal facing surface which engages a corresponding distal facing surface of the dosing assembly, when the container is in operational contact with the dosing assembly. Moreover, at least one of the proximal and distal facing surfaces may define one or more of the coding surfaces.

As an example the at least one of the proximal facing surface(s) and the distal facing surface(s) may define at least one axially extending protrusion, which may be adapted to engage a corresponding axially extending indentation defined in the other one of the proximal and distal facing surface when the dosing assembly is in operational contact with the container. Moreover, one or more of the at least one axially extending protrusion and corresponding indentation may define one or more of the coding surfaces.

In one embodiment the driver of the dosing assembly engages a dose setting member, when the container is in operational contact with the dosing assembly. The driver of the dosing assembly may engage the dose setting member which may be connected to the piston rod such that that rotation of the dose setting member e.g. means of the driver, causes the dose setting member to rotate whereby the dose setting member is moved in the proximal direction relative to the piston rod and a dose is set. In one embodiment the outer surface of the piston rod is threaded and the dose setting member defines a passage having a threaded inner surface adapted to engage the threaded outer surface of the piston rod. When the container is fastened to the dosing assembly, the driver and the dosing assembly may be locked for relative rotation e.g. by means of the abovementioned radially extending depressions/projections, whereby rotation of the driver causes the dose setting member to rotate and a dose to be set.

In one embodiment, the driver encircles the dose setting member when the container is attached to the dosing assembly, and, thus, an inner surface of the driver is adapted to engage an outer surface of the dose setting member in order to lock the driver and the dose setting member for relative rotation. Alternatively, the dose setting member encircles the driver when the container is attached to the dosing assembly, and, thus, an inner surface of the dose setting member is adapted to engage an outer surface of the driver in order to lock the driver and the dose setting member for relative rotation.

As an example at least one of the driver and the dose setting member may define at least one radially extending projection, each of the at least one projection engages corresponding depression(s) defined in the other one of the driver and the dose setting member when the dosing assembly is in operational contact with the container. Moreover, at least one of the coding surfaces may be defined by the at least one projection and corresponding depression.

In some embodiments, the container and the dosing assembly is adapted to be mutually fastened and locked through a relative axially translational and non-rotational movement.

In one embodiment the second fastening means of the container defines a radially extending groove and the first fastening means of the dosing assembly comprises a locking member which is movable between: a locking position wherein at least a part of the locking member engages at least a part of the radially extending groove whereby the container is locked for translational movement relative to the dosing assembly; and a non-locking position wherein the locking member does not engage the radially extending groove whereby the container is not locked for translational movement relative to the dosing assembly.

The radially extending groove may be defined on the outer surface of the container and may extend around the entire circumference of the container so as to define a circumferentially extending groove. Alternatively, the radially extending groove may be split into groove parts spaced along the outer circumference of the container. In one embodiment the locking member is adapted to engage a part of the groove during locking/fastening of the container to the dosing assembly. In another embodiment the locking member is adapted to engage the entire groove during locking/fastening of the container to the dosing assembly.

In order to change the locking member between the locking position and the non-locking position, the locking member may be adapted to change from a locking shape wherein the locking member during use is positioned in the locking position to a non-locking shape wherein the locking member during use is positioned in the non-locking position. In one embodiment the locking member is adapted to change from the locking shape to the non-locking shape be application of finger pressure to predetermined areas/positions on the locking member, and in the reverse direction when the finger pressure is removed.

In one embodiment the locking member comprises a resilient material biasing the locking member towards the locking position. The resilient material may be any material suitable to be reshaped by application of finger pressure, such as rubber, soft plastics, spring steel. In one embodiment the locking member comprises at least two buttons which when the locking member is assembled in the dosing assembly are accessible from an outer surface of the dosing assembly. Application of finger pressure to the buttons (such that the two buttons are moved towards each other) causes the locking member to change from the locking to the non-locking shape whereby a user may remove the container from the dosing assembly. Moreover, at least a part of the locking member may be substantially elliptical when defining the locking shape and substantially cylindrical when defining the non-locking shape.

The container may comprise a cartridge for accommodation of the medicament, such as a standard glass cartridge. The cartridge may be detachable attached to a cartridge holder. In such case, the cartridge holder constitutes the container. A user may replace an empty cartridge with a full cartridge. Alternatively, the cartridge may be non-detachably attached to the cartridge holder, such that cartridge holder is deformed/damaged if the cartridge is removed. The cartridge holder may be integrated with or attached to the proximal end part of the container. In one embodiment the cartridge holder is retained axially relative to the proximal end part, whereas the cartridge holder and the proximal end part are allowed to rotate relative to each other. In such an embodiment, the container coding surface may interlock with the dosing assembly coding surface but due to the distal part of the container being rotatable relative to the container coding surface, the risk of destructive rotational forces acting on the coding surfaces is reduced. In yet another embodiment, the cartridge holder and the proximal end part define a monolithic element i.e. forming one element without seams. In yet a further embodiment the proximal end part, the cartridge holder and the cartridge define a monolithic element.

In a FOURTH aspect the present invention relates to a container suitable for use in a medical delivery system according to the first, second or third aspect of the invention.

The invention according to the fourth aspect may comprise any feature and/or element of the invention according to the first, second or third aspect of the invention.

In a FIFTH aspect the present invention relates to a dosing assembly suitable for use in a medical delivery system according to the first, second or third aspect of the invention.

The invention according to the fifth aspect may comprise any feature and/or element of the invention according to the first or third aspect of the invention.

### BRIEF DESCRIPTION OF THE DRAWINGS

The invention will now be described in further detail with reference to the drawings in which:

FIGS. 1-4 disclose a resilient locking member according to a first embodiment and relating to the second aspect of the invention,

FIGS. 5 and 6 disclose a container according to a second embodiment and relating to the third aspect of the invention,

FIG. 7 discloses a medical delivery system according to the second embodiment,

FIG. 8 discloses a dosing assembly according to the second embodiment,

FIG. 9 discloses a proximal part of a container according to the second embodiment,

FIGS. 10-13 disclose a dose setting member according to the second embodiment,

FIGS. 14a-15 disclose a medical delivery system according to a third embodiment and relating to the first aspect of the invention, and

FIGS. 16-18 disclose different embodiments of the coding surfaces of the second embodiment.

### DETAILED DESCRIPTION OF THE DRAWINGS

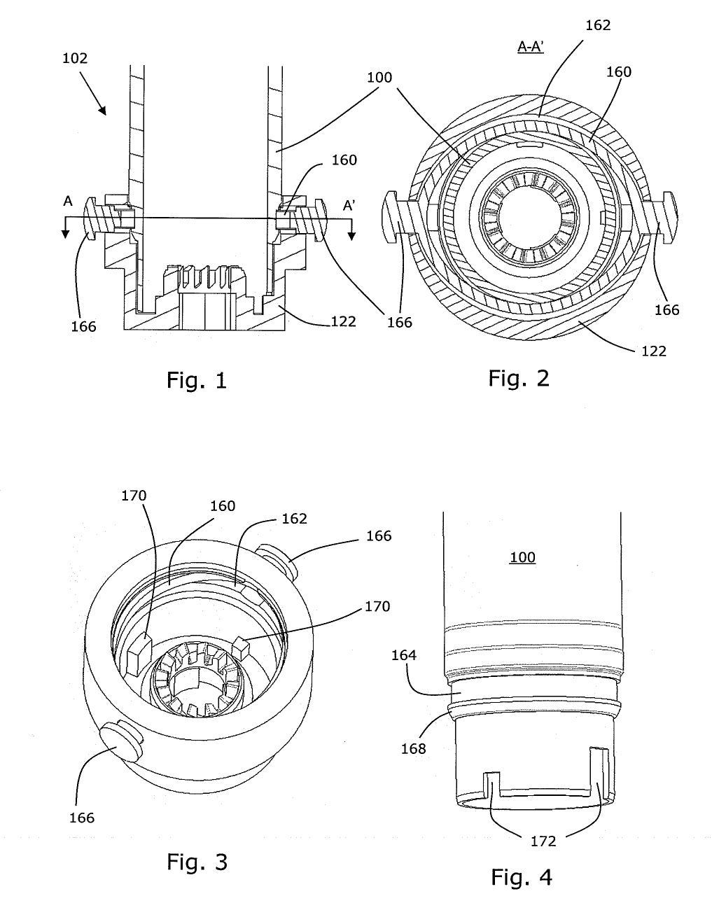

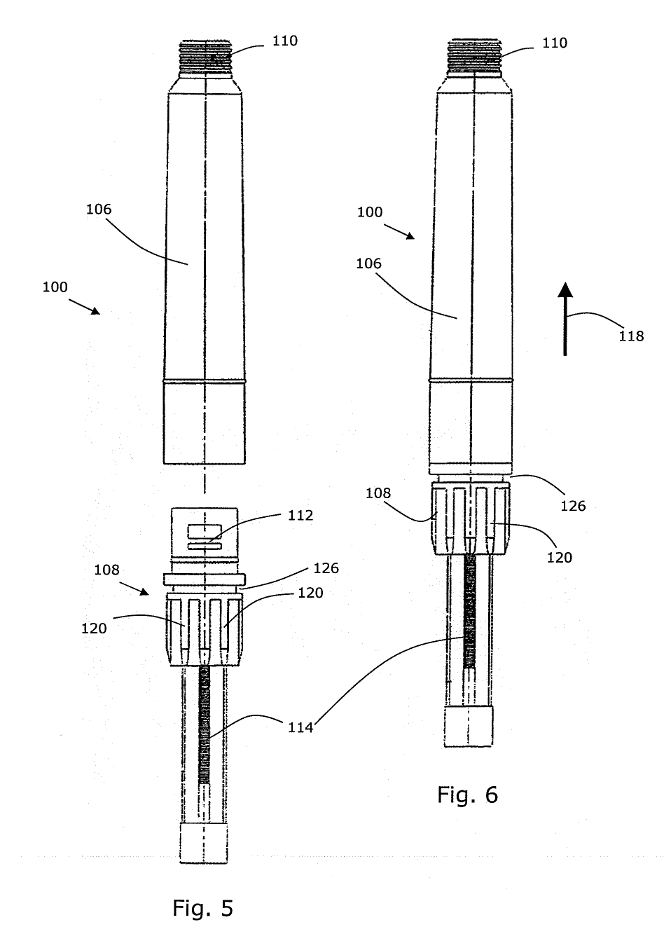

FIGS. 1-4 disclose a container 100 for a medical delivery system 102 comprising a dosing assembly 122 (only shown in part). The container may comprise a cartridge as described below in relation to FIGS. 5-18. The cartridge may be accommodated in a cartridge holder. In the embodiment of FIGS. 1-4 the dosing assembly 122 comprises a locking member 160, received in a circumferentially extending recess 162 of the dosing assembly 122. The locking member 160 comprises a resilient material allowing the locking member 160 to change shape from a locking shape wherein at least a part of the locking member engages a radially extending groove 164 of the container 100, to a non-locking shape wherein the locking member 100 does not engage the radially extending groove 164 of the container 100. Then the locking member 160 is in its non-locking shape, the container 100 may be removed from the dosing assembly.

Due to the resilient material a user may change the shape of the locking member 160 by application of finger pressure to two buttons 166 which are positioned opposite each other. When the two buttons 166 are pressed towards each other, the locking member 160 changes shape from the locking position towards/into the non-locking position. When the locking member 160 is in its locking position a part of the member 160 defines a substantially elliptical shape (shown in FIGS. 2-3) which, when the two buttons are forced towards each other, is changed to a substantially circular shape. The two buttons 166 are accessible from an outer surface of the dosing assembly 122.

The container 100 comprises an inclined surface 168 which allows the container 100, when forced into the dosing assembly 122, to move the locking member 166 away from the locking position, and, thus, allowing the container to be inserted into the dosing assembly 122. The inclined surfaces allow the user to fasten the container 100 to the dosing assembly 122 without application of pressure to the buttons 166. Thus, in embodiments without the inclined surface 166, a user must move the locking member 160 from the locking position to the non-locking position by application of finger pressure to the buttons 166 in order to be able to fasten the container to the dosing assembly.

In FIG. 4, the dosing assembly comprises two axially extending protrusions 170 which prevent a container 100 from being fastened to the dosing assembly 122 unless the container 100 defines corresponding indentations 172. Accordingly, the indentations 172 and the protrusions 170 define coding surfaces of the device. In particular embodiments, the proximal portion of the container forming the depressions and projections is formed so as to be able to rotate relative to the remainder of the container. In another embodiment (not shown) the locking member 160 defines one or more radially extending projections adapted to be received in corresponding radially extending depressions of the container when the container is fastened to the dosing assembly. Accordingly, containers not defining the depressions cannot be fastened to the dosing assembly. The shape and position of the depressions and the projections may be varied in order to define different coding geometries. The geometries may be changed by varying the circumferential extent and/or the circumferential position and/or the shape of the projections/indentations.

Moreover, the container and the dosing assembly according to the first aspect of the invention may comprise any of the coding geometries described in relation to the third aspect of the invention.

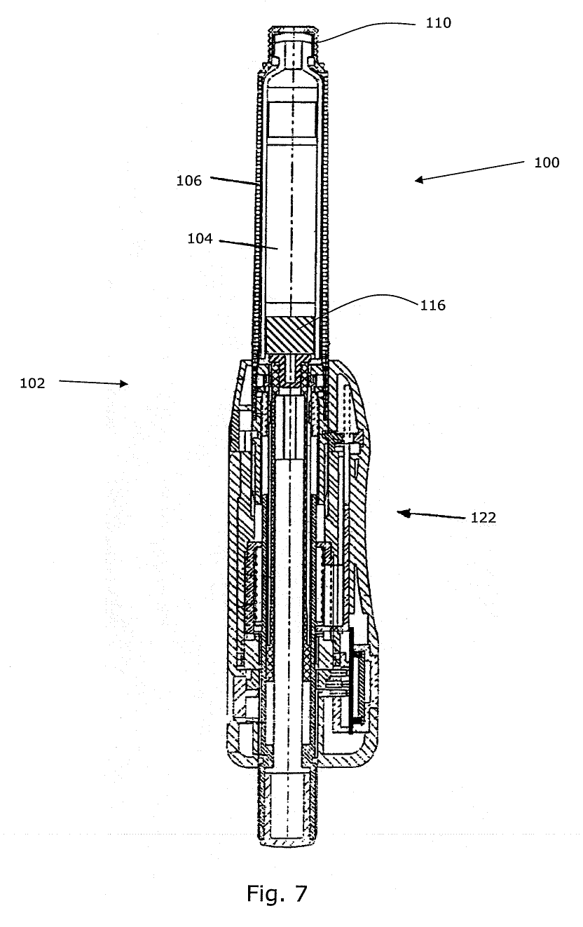

FIGS. 5-13 disclose the invention according to the third aspect of the invention. FIGS. 5 and 6 disclose container 100 for a medical delivery system 102 (cf. FIG. 7) comprising a dosing assembly 122. The container 100 comprises a cartridge 104 (not visible in FIGS. 5 and 6) accommodated in a cartridge holder 106. A proximal end part 108 is provided in a proximal end of the container 100. FIG. 5 discloses the container prior to attachment of the proximal end part 108 where the cartridge may be inserted into the cartridge holder 106. FIG. 6 discloses the container after the proximal end part 108 has been non-detachably attached to the cartridge holder 106. The cartridge holder 106 comprises threaded distal end 110 for fastening a hub with a cannula (not shown) to the container. The proximal end part 108 comprises fastening means 112 adapted to engage corresponding fastening means (not shown) of the cartridge holder 106 so as to fasten the proximal end part 108 to the cartridge holder 106.

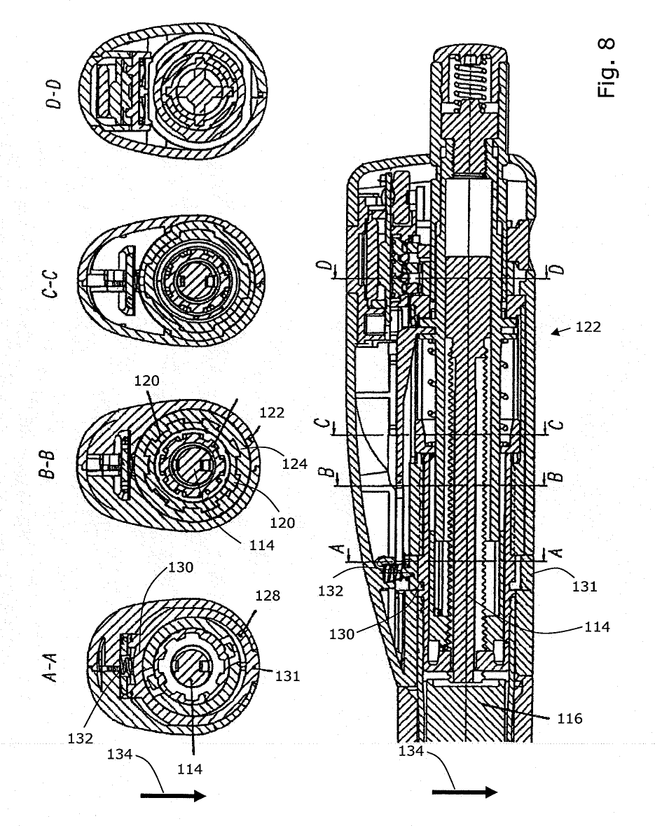

Moreover, the container 100 comprises a piston rod 114 which when the proximal end part 108 is attached to the cartridge holder 106 abuts a piston 116 (cf. FIGS. 7 and 8) such that upon distal movement of the piston rod 114, the piston 116 is moved in a distal direction 118 whereby a dose of the medicament contained in the cartridge 104 may be expelled, through a cannula. The proximal end part 108 comprises eight radially extending projections 120 which are used to lock the proximal end part 108 for rotation relative to the dosing assembly 122 (shown in FIGS. 7 and 8), when said radially extending projections engage corresponding radially extending depressions 124 of the dosing assembly 120. The radially extending projections 118 further serves the purpose of defining coding surfaces, as the radially extending projections 118 are provided in a predetermined pattern and define predetermined shapes (radially, axially and circumferentially), and, thus, only containers having radially extending projections 118 provided in a predetermined coding pattern may be fastened to a specific dosing assembly 122.

The proximal end part 108 comprises a circumferentially extending second fastening means 126 in the form of a groove adapted to be engaged by a first fastening means 128 of the dosing assembly 122. The first fastening means 128 comprises a radially extending projection 130, which is movable in and out of at least a part of the second fastening means 126 of the container 100 upon activation of a button 131, whereby the container 100 is locked and unlocked, respectively, to/from the dosing assembly 122. The first fastening means 128 comprises a spring 132 arranged to bias the radially extending projection 130 in a first direction 134. Upon insertion of the proximal end part 108 into the dosing assembly 122, an inclined surfaces 136 of the proximal end part 108 forces the first fastening means 128 in a direction opposite the first direction 134. When the second fastening means 126 is in the same axial position as the first fastening means 128, the spring 132 forces the radially extending projection 130 into the groove of the second fastening means 126 of the container, whereby the container is locked axially to the dosing assembly. In order to unlock the container 100 from the dosing assembly 122, the button 131 must be moved in a direction opposite the first direction 134, whereby the radially extending projection 130 is moved out of the groove of the second fastening means 126. In one embodiment the first and/or second fastening means comprises one or more coding surfaces, whereby only predetermined containers 100 can be fastened to predetermined dosing assemblies 122.

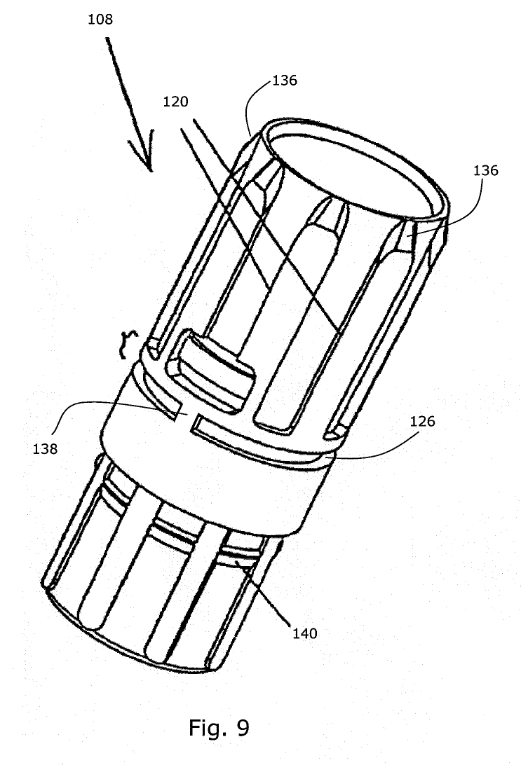

FIG. 9 discloses the proximal end part 108 comprising the eight radially extending projections 120 each of which has an inclined surface 136 as described above. In other embodiments the proximal end part 108 may comprise fewer or further radially extending projections. The radially extending projections define coding surfaces for ensuring that only predetermined containers are attached to predetermined dosing assemblies. The coding surfaces may varied by changing the circumferential, the radial or the axial extent of the projections 120 or by providing the projections in a predetermined pattern circumferentially, such as in a symmetrical or an asymmetrical pattern.

In the embodiment of FIG. 9 the second fastening means 126 of the proximal end part forms part of the coding of the container/proximal end part due to the coding projection 138. Accordingly, only dosing assemblies having a corresponding coding indentation can be fastened to the container. In other embodiments of the proximal end part 108, the axial extent of the second fastening means 126 are varied in order to code the container/proximal end part. In yet other embodiments, the radial depth of the second fastening means 126 is used for the coding of the device.

In order to fasten the proximal end part 108 to the cartridge holder 106 the proximal end part 108 comprises fastening means 140 adapted to engage corresponding fastening means of the cartridge holder 106 so as to non-releasably attach the cartridge holder 106 and the proximal end part 108 to each other.

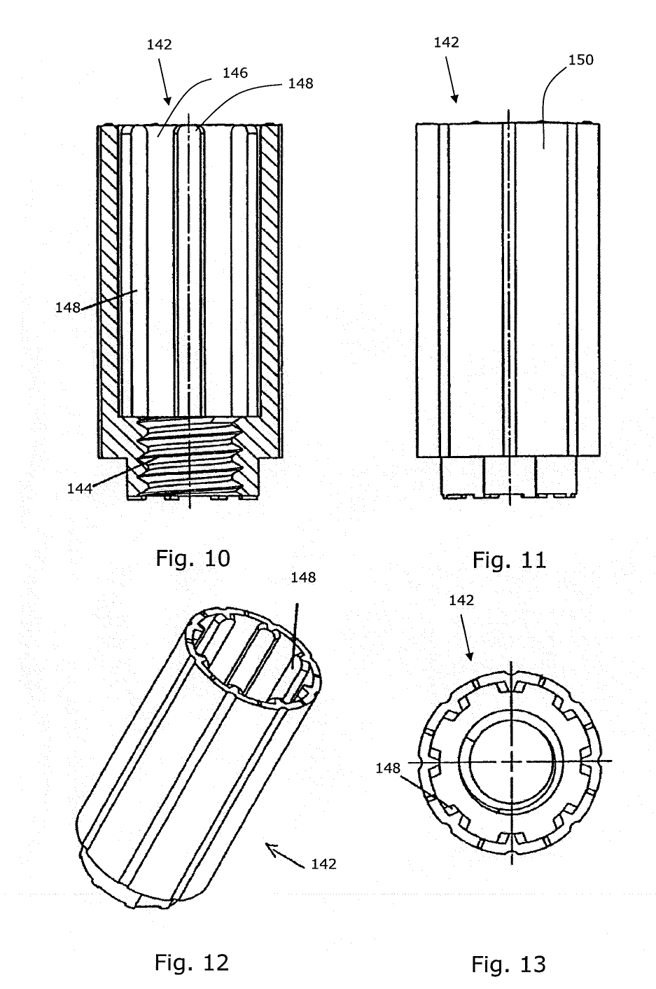

FIGS. 10-13 disclose the dose setting member 142 which has a threaded inner surface 144, adapted to engage the threaded outer surface of the piston rod 114. The dose setting member 142 is adapted to be retained rotationally relative to a driver (not shown) of the dosing assembly 122 due to engagement between the radially extending indentations 146 defined on an inner surface 148 of the dose setting member and radially extending projections (not shown) of the driver. In some embodiments the radially extending indentations 146 are defined on the outer surface 150 of the dose setting member 142 and the driver is designed to engage the indentations 146 of the outer surface of the dose setting member 142. At least a part of the coding surfaces may be defined by the radially extending indentations 146, the geometry of which may be varied by changing one or more of the circumferential width, the radial depth, the axial length and the circumferential position of the indentations.

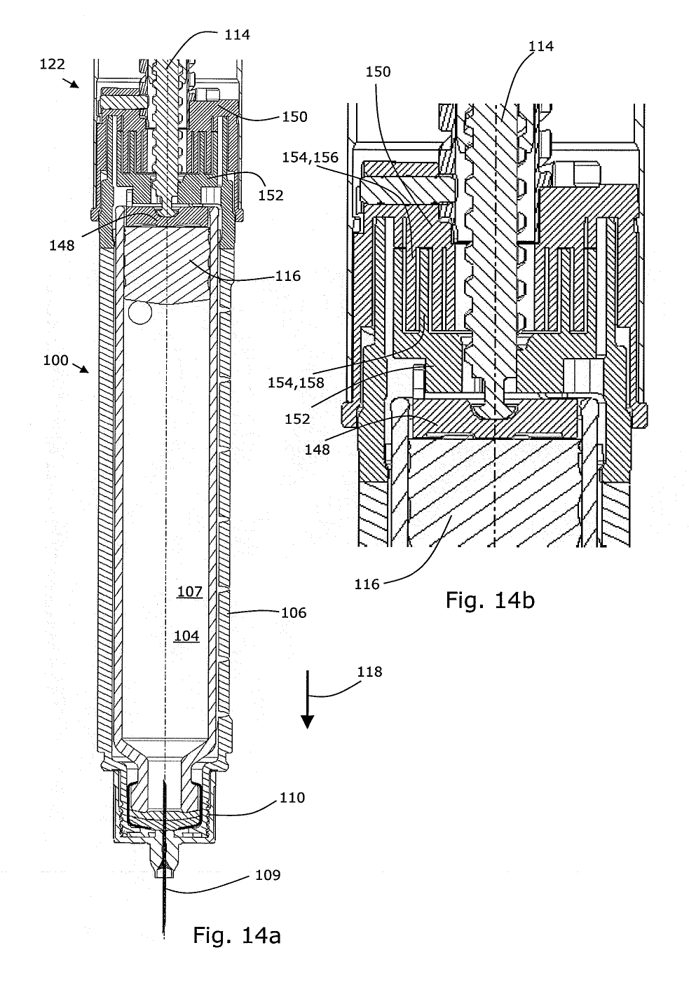

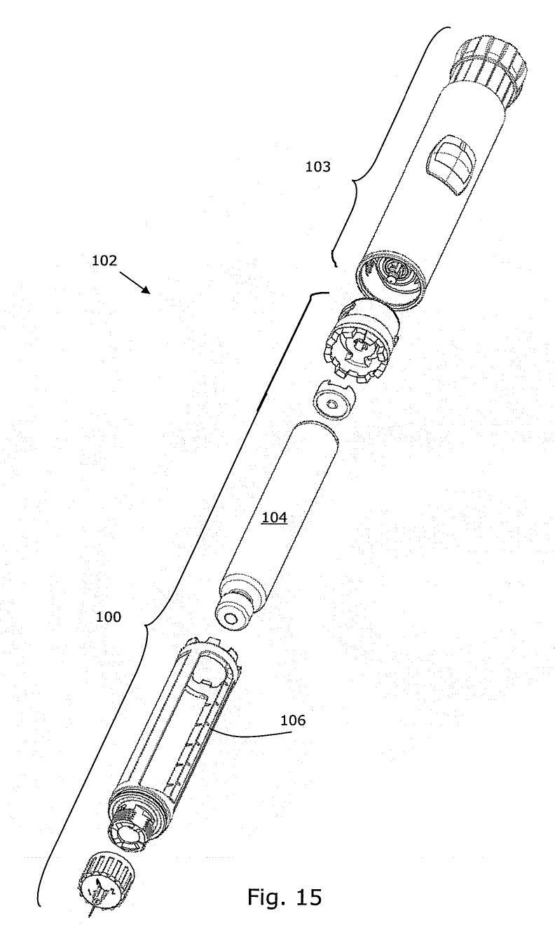

FIGS. 14a-15 disclose a medical delivery system 102 according to a third embodiment of the invention. The medical delivery system 102 comprises a container 100 and a dosing assembly 122. The container comprises a cartridge holder 106 which accommodates a cartridge 104 defining a chamber 107 for accommodation of a medicament. A piston 116 is provided in the chamber 107, and when the piston is moved in the distal direction 118, a dose of the medicament provided in the chamber 107 is expelled through a cannula 109 which may be screwed onto the cartridge holder 106 via the threaded distal end 110. The container comprises a proximal end part 108 which is connected to the cartridge holder 106.

The dosing assembly 122 comprises a piston rod 114 which when the container 100 is attached/fastened to the dosing assembly 122, abuts the piston 116 via an interconnecting element 148 which is more rigid than the piston 116, which must be flexible enough to provide a sealing effect between the piston 116 and an inner sidewall of the cartridge 104. By providing the interconnecting element the force provided by the piston rod 114 during dosing is applied to a larger area of the piston than if the piston rod abutted the relatively soft piston directly.

The dosing assembly 122 and the container 100 comprises a first and second coding element 150,152, each of which comprises a plurality of axially extending ring-shaped coding protrusions 154. The first coding element 150 comprises distally extending coding protrusions 156 and the second coding element 152 comprises proximally extending coding protrusions 158. The coding protrusions may have different lengths and widths. Accordingly, only containers with predetermined second coding elements 152 may be attached/fastened to dosing assemblies with predetermined first coding elements 154. In the system of the embodiment of FIG. 14b the first and second coding elements 150,152 may each have up to five concentric coding protrusions. However, in the drawing the first coding element 150 comprises only three coding protrusions while the second coding element comprises only two coding protrusions. It will be appreciated, that in other embodiments the total number of ring-shaped protrusions are different such as one, two, three, four, six, seven, etc.

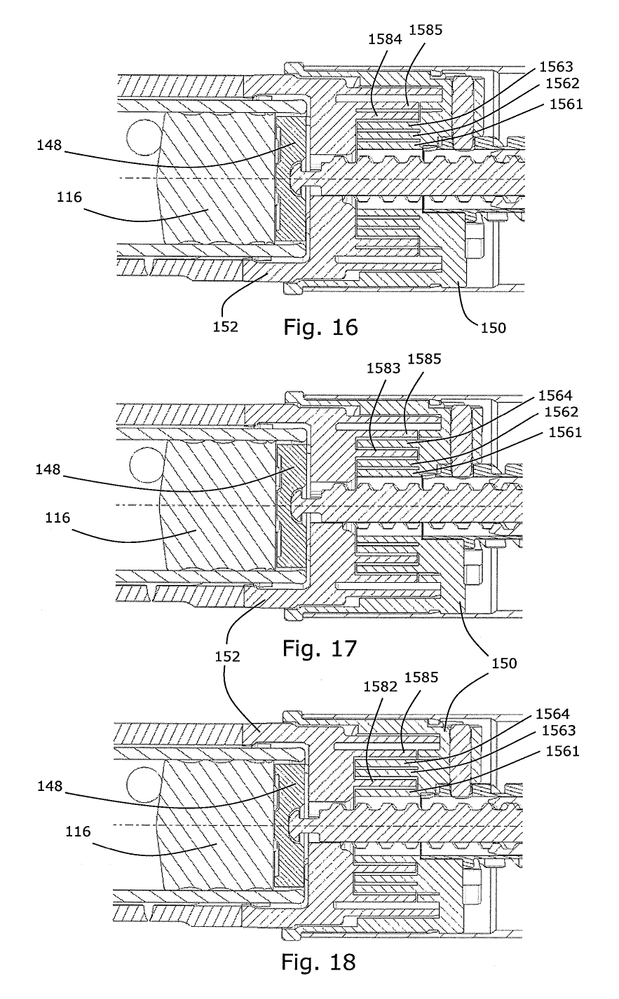

FIGS. 16-18 discloses different embodiments of the first and second coding elements 150,152. It will be appreciated that although each of the first coding elements 150 in the three figures each have three distally extending coding protrusions, and each of the second coding elements 152 have two proximally extending coding protrusions, any of the three containers can only be mated with/attached to a predetermined dosing assembly.

In FIG. 16 the first coding element 150 comprises three distally extending coding protrusions 1561,1562,1563 and the second coding element 152 comprises two proximally extending coding protrusions 1584,1585.

In FIG. 17 the first coding element 150 again comprises three distally extending coding protrusions 1561,1562,1564 and the second coding element 152 comprises two proximally extending coding protrusions 1583,1585.

In FIG. 18 the first coding element 150 comprises three distally extending coding protrusions 1561,1563,1564 and the second coding element 152 comprises two proximally extending coding protrusions 1582,1585.

As described above, the axially extending protrusions may be formed as ring-shaped structures being arranged to coaxially mesh and axially overlap when the container is properly locked to its associated dosing assembly. The ring-shaped configuration of a particular container forms ring-shaped axial indentations at radial positions in between or next to a neighbouring ring-shaped axial protrusion. At the radial position of a ring-shaped indentation, a corresponding ring-shaped axial protrusion are positioned in the dosing assembly which is intended for use with the particular choice of container.

For a particular container, the protrusions which provide the ring-shaped configurations may be formed as continuous ring-shaped elements extending through 360 degrees. However, it is to be noted that, within the context of the present invention, the axially extending protrusions may be formed by one or more segments arranged at the particular radial position but having only a small angular extension. As long as non-compatible dosing assemblies are provided with a full circular ring-shaped axial protrusion formed at the same radial position as the said one or more segments of the particular container, the use of such container with the non-compatible dosing assemblies is effectively prevented.

It should be noted that while the embodiment shown in FIG. 15 indicates that at least one projection extends radially outwards for forming a bayonet coupling between the container and the dosing assembly, all the embodiments shown in FIGS. 14a-18 may comprise any coupling mechanism for locking the container to the dosing assembly. For example, the coupling mechanisms described having regard to the embodiments shown in FIGS. 1-13 may be used. In such case, the container may include a circumferential groove forming the second fastening means. Alternatively, a threaded coupling may be used for coupling the container with the dosing assembly.

1. A medical delivery system comprising:
a container adapted to contain a medicament in a chamber defined by the container and a slidably arranged piston which is axially moveable in a distal direction towards an outlet so as to reduce the volume of the chamber and expel the medicament through the outlet, the container comprising a housing section having a proximal cylindrical rim section which defines a proximal internal opening;
a piston rod adapted to extend through said proximal internal opening of the container and move the piston in the distal direction;
a dosing assembly adapted to be fastened to the container so as to bring the dosing assembly into operational contact with the container, the dosing assembly comprising a driver adapted to move the piston rod in the distal direction when the dosing assembly is in said operational contact with the container;
wherein the dosing assembly defines a first fastening means adapted to engage a second fastening means of the container so as to lock the container in said operational contact with the dosing assembly;
wherein the proximal end part of the container defines at least one coding surface dedicated the medicament to be contained in the container, the coding surface comprising one or more proximally extending protrusion(s), the or each proximally extending protrusion(s) being arranged in a first ring configuration concentric with said rim portion;
wherein the dosing assembly defines a coding surface dedicated the medicament to be contained in the container and comprising one or more distally extending protrusion(s), the or each distally extending protrusion(s) being arranged in a second ring configuration; and
wherein the first and the second ring configurations are adapted to mesh and axially overlap when the container is locked in operational contact with the dosing assembly.

2. A medical delivery system comprising:
a first container according to claim 1, the container being adapted to be fastened to a first dosing assembly according to claim 1,
a second container according to claim 1, the container being adapted to be fastened to a second dosing assembly according to claim 1,
wherein the first and/or the second coding mechanism of at least two of the first container, the second container, the first dosing assembly and the second dosing assembly, is/are adapted to prevent the first dosing assembly and second container from being fastened to each other, and to prevent the second dosing assembly and the first container from being fastened to each other.

3. A medical delivery system according to claim 1, wherein the first container is coupled to the first dosing assembly through a predetermined movement which is substantially identical to the predetermined movement required for coupling the second container to the second dosing assembly.

4. A medical delivery system according to claim 1, wherein the container and the dosing assembly are configured to allow relative rotation between a distal end portion of the container and the dosing assembly irrespective of the mutual axial distance during fastening of the container to the dosing assembly.

5. A medical delivery system according to claim 1, wherein the container and the dosing assembly is adapted to be mutually locked by a movement comprising a relative rotational movement.

6. A medical delivery system according to claim 1, wherein the container and the dosing assembly is adapted to be mutually locked through a translational and non-rotational movement.

7. A medical delivery system according to any of claims 6, wherein the second fastening means of the container defines a radially extending groove and the first fastening means of the dosing assembly comprises a locking member which is movable between
a locking position wherein at least a part of the locking member engages at least a part of the radially extending groove whereby the container is locked against translational movement relative to the dosing assembly, and
a non-locking position wherein the locking member does not engage the radially extending groove whereby the container is not locked for translational movement relative to the dosing assembly.

8. A medical delivery system according to claim 7, wherein, by application of finger pressure to predetermined areas of the locking member, the locking member is adapted to change shape from a locking shape wherein the locking member during use is positioned in the locking position to a non-locking shape wherein the locking member during use is positioned in the non-locking position.

9. A medical delivery system according to claim 8, wherein at least a part of the locking member is substantially elliptical when defining the locking shape and substantially cylindrical when defining the non-locking shape.

10. A medical delivery system according to claim 6, wherein one of the first and second fastening means defines one or more radially extending projection(s) which during fastening is adapted to be received in one or more corresponding radially extending depressions of the other one of the first and second fastening means.

11. A medical delivery system according to claim 1, wherein the one or more axially extending protrusions of the first ring configuration of the container is/are formed in the proximal opening of the container.

12. A medical delivery system according to claim 1, wherein the container is adapted to hold a glass cartridge accommodating the piston and wherein the proximally extending protrusion(s) forming the first ring configuration is/are formed by an element distinct from the glass cartridge.

13. A container suitable for use in a medical delivery system according to claim 1.

14. A dosing assembly suitable for use in a medical delivery system according to claim 1.
